(19) United States
(12) Patent Application Publication
(10) Pub. No.: US 2010/0216249 A1
(43) Pub. Date: Aug. 26, 2010

(54) DNA-Based Biosensors

(75) Inventors: Achillefs Kapanidis, Oxford (GB); Konstantinos Lymperopoulos, Oxford (GB); Michael Heilemann, Bielefeld (DE)

Correspondence Address: SPECKMAN LAW GROUP PLLC, 1201 THIRD AVENUE, SUITE 330, SEATTLE, WA, 98101, US

(73) Assignee: ISIS INNOVATION LIMITED, Oxford (GB)

(21) Appl. No.: 12/525,721

(22) PCT Filed: Feb. 12, 2008
(86) PCT No.: PCT/GB2008/000488

§ 371 Date: Dec. 15, 2009

(30) Foreign Application Priority Data
Feb. 12, 2007 (GB) 0702681.8

(51) Int. Cl. G01N 33/68

(52) U.S. Cl. 436/86

(57) ABSTRACT

There is described a method of detection of the protein-dependent coincidence of DNA in a sample which comprises detection using luminescence of one or more luminophores introduced into DNA with one, two or more DNA fragments which fragments are bound using one or more DNA-binding proteins.

This invention relates to a novel fluorescence spectroscopy method and assay platform for the detection of, inter alia, DNA-binding proteins and their ligands and to sensors related thereto.

DNA-binding proteins constitute a large family of proteins with diverse and important biological functions. DNA-binding proteins include important gene-regulatory proteins known as transcription factors and DNA-processing proteins (such as DNA and RNA polymerases, DNA ligases, DNA endonucleases and exonucleases, and DNA repair and recombination proteins). Such proteins usually recognise and bind with high affinity to specific sequences within DNA. Although the methods described in this application are applicable to any sequence-specific DNA-binding protein, hereafter we will focus our attention to transcription factors, a specific family of DNA-binding proteins.

Transcription is the process during which the genetic information encoded on the DNA is transferred on another nucleic acid, RNA; it is characteristic that most of the control of gene expression occurs at the level of transcription. Transcription factors are proteins that bind to specific DNA regions (promoter, activating, operator or enhancer regions) and regulate the transcription of a gene; approximately 6% of all human genes code for transcription factors. Transcription factors may enhance or obstruct the association of RNA polymerase with promoter DNA and modify the amount of newly synthesised RNA; transcription factors can also be selectively activated or deactivated by other proteins or small molecules. Since transcription factors play such a central role in gene expression, changes in their cellular concentrations controls fundamental biological processes such as development and cell-commitment. Therefore, the detection and quantitation of transcription factors can provide essential information about gene regulation

Transcription factors are also the endpoints of signal-transduction cascades, acting as indirect sensors of the extracellular environment. Given the central role of transcription factors, it is not surprising that alterations in their concentration levels or in their activity (e.g., due to mutations) can lead to a variety of diseases. For example, the alteration of the activity of many transcription factor in humans can lead to cancer and is associated with many diseases and neurodegenerative disorders; it is notable that alterations in the activity of transcription factor p53 is involved in 50% of all human cancers. Moreover, for a single transcription-factor family only (nuclear receptors), there are currently at least 30 drugs at various stages of development. Therefore, since the concentration levels of transcription factors differ at diseased states of a living cell, detecting profiles of transcription factors can be extremely useful for biomedical purposes such as sensitive diagnostics and drug discovery. These capabilities become more powerful when low protein concentrations are detectable. Finally, establishing assays that monitor the modulation of the DNA-binding activity of transcription factors by small molecules is crucial for drug discovery.

Transcription factors can aid in the detection of small molecules (also referred to as “ligands”). Since transcription factors often have a ligand-binding domain in addition to the DNA-binding domain, they can be used as natural biosensors that detect the concentration of these ligands. Ligands are small molecules, often metabolites and are critical for the function of a transcription factor. Examples of transcription factors that recognise specific ligands include the nuclear Liver X receptor (LXR) which is a glucose sensor, the bacterial catabolite activation protein (CAP) which is activated by cyclic AMP (cAMP) and the bacterial NikR repressor which is regulated by nickel ions. Understanding the mechanism of activation of a transcription factor by its respective ligand is crucial for understanding gene regulation. Moreover, the detection of small metabolites (glucose, cAMP, glutamine, toxic metals, etc) can aid the analysis of biological and environmental samples.

Given the importance of DNA-binding proteins and especially of transcription factors, several methods exist for their detection and quantification. A first group of methods involve electrophoretic mobility shift assays (EMSAs or “gel-shifts”), DNA footprinting assays, enzyme-linked immunosorbent assay (ELISA) assays.

EMSA assays are based on the observation that the protein-DNA complex migrates slower than the free DNA in non-denaturing polyacrylamide gels. A variation of the basic EMSA assay is “supershift-EMSA”, wherein an antibody against the transcription factor recognizes and binds to the protein-DNA complex and thus migrates slower than the simple binary protein-DNA complex.

DNA footprinting assays are based on the fact that a DNA-binding protein will protect its DNA-binding site from cleavage from enzymatic or chemical cleavage. The DNA is usually radioactively end-labelled, so upon gel electrophoresis, the cleavage pattern can be detected. The cleavage pattern of the DNA fragment will be different if a DNA-binding protein is bound on the fragment thus protecting it from cleavage.

In ELISA assays for DNA-binding proteins, an antibody specific for the transcription factor is immobilised on a surface and the transcription factor binds to it. A second specific antibody for the particular protein is used to bind to the immobilized DNA-binding protein. The second antibody is linked with an enzyme that upon binding with its substrate provides a colorimetric or luminescence signal.

The aforementioned methods are tedious, time-consuming, expensive, not amenable for high-throughput detection and are often only of qualitative nature. Gel-shift assays and DNA footprinting also often require use of radioactive reagents and of acrylamide (a powerful neurotoxin). Moreover, the aforementioned assays cannot be used for detecting low-abundance DNA-binding proteins.

Use of microplates and microarrays for transcription-factor detection assays such as ELISA are improvements over the aforementioned assays since they introduce high-throughput formats, reduced tedium and better detection sensitivities. However, the amount of sample required is still very significant; the assays require immobilisation of antibodies or DNA fragments on modified solid supports (which add cost for special plates or arrays), as well as medium-to-high end instrumentation for recording images or light intensities; often, there are several steps for sample preparation and treatment with various enzymes and chemicals along with several washes (separation steps) and incubations; finally, there is often a need for signal amplification. This need is well aligned with an overall move in diagnostics towards assays that do not require analyte amplification, thus reducing the cost, tedium and time that amplification requires.

Prior to 2002, several fluorescence assays were used to detect DNA-binding proteins; these assays include fluorescence quenching upon protein-DNA binding; fluorescence polarisation increase upon protein-DNA binding; and fluorescence resonance energy transfer (FRET; a proximity-based assay, since the energy-transfer efficiency depends on the distance between two fluorophores, a donor and acceptor). These fluorescence assays had the advantage of being homogeneous (no separation steps), solution-based and sensitive, but since there were not generally applicable, they were not widely adopted.

In 2002, an improved ensemble-fluorescence-based method (in which DNA fragments are used as “molecular beacons”) which detects DNA-binding proteins without separation steps was described. With reference particularly to U.S. Pat. No. 6,544,746, published in 2003, there are generally described methods of detecting and quantifying specific proteins, in particular sequence-specific DNA-binding proteins, based upon proximity-based luminescence transfer. Two double-stranded oligonucleotides having short (˜6 nucleotides) fully complementary overhangs are isolated and by combining the two double-stranded oligonucleotides, a complete DNA element is formed across the juncture of the oligonucleotides. The first oligonucleotide is labelled with a fluorophore (a fluorescent donor) and the second oligonucleotide is labelled with a fluorescence-quenching molecule (fluorescent acceptor). In the absence of the protein specific to the two oligonucleotides, the complementarity between the short overhangs alone is insufficient to produce a stable full-sequence binding site; however, in the presence of the protein that recognizes the full DNA binding site, the transiently formed sites become stable and the two oligonucleotides stay in close proximity. When the two oligonucleotides are in close proximity, the fluorescent donor of the first oligonucleotide transfers (through FRET) its excited-state energy to the fluorescent acceptor of the second oligonucleotide, ultimately resulting in the quenching of the emitted light from the fluorescent donor. The quenching of the fluorescent signal is correlated with the association of the DNA binding factor to the cognate DNA element. This concept was also used for sensing the concentration of small molecules that bind to transcription factors, such as cAMP.

The method is compatible with oligonucleotides affixed to a solid phase substrate, such as, for example, a microtiter plate, microarray slide, membrane or microsphere. These strategies are compatible with multiplexing and with the use of a single fluorophore that is attached away from the DNA binding site. However, use of arrays requires a significant volume of cellular extract, additional separation steps, instrumentation and consumables, as well as washes and incubations that might result in dissociation of any weak protein-DNA complexes (thus precluding protein detection). Moreover, the sensitivity of the assay is still not adequate for low-abundance proteins. Finally, solid-phase-based assays are incompatible with direct, real-time monitoring DNA-binding proteins in single cells.

Whilst U.S. Pat. No. 6,544,746 includes a disclosure that the oligonucleotide pairs may be free to diffuse in solution, a particular disadvantage of the approach described therein is that due to the reliance upon proximity-based luminescence transfer, there is a strict requirement for fluorophore proximity which constrains fluorophores to be close to the protein-DNA interface; this location of the fluorophores may inhibit or reduce protein-DNA binding due to steric hindrance, limiting the method to specific cases with existing detailed structural understanding of the protein-DNA complex. Moreover, the solution-based embodiment cannot easily detect more than a few (i.e. 3 to 4) DNA-binding proteins simultaneously (i.e., in the same detection volume); parallel detection will inevitably require the need of microarrays. In addition, the sensitivity of ensemble fluorescence is not adequate for reaching low protein concentrations (10-1000 pM); we note that the lowest possible protein concentration in a bacterial cell is ˜1 nM (1 protein copy per bacterial cell) and for a eukaryotic cell is ˜20 pM (1 protein copy per nucleus); therefore, the ensemble molecular-beacon assay cannot detect low-abundance proteins. Finally, the ensemble assay addresses cellular extracts and has not been extended for real-time monitoring of DNA-binding proteins in living cells.

Single-molecule fluorescence spectroscopy (SMFS), a family of microscopy-based methods that detect molecules in small volumes (attoliter-femtoliter) and low analyte concentrations (pM-nM), is currently revolutionizing many areas of chemistry and biology. Since SMFS is compatible with high-throughput formats, it also represents a rapid and affordable way to detect biomolecules at low amounts and concentrations. While static and dynamic heterogeneity are well-known complications in ensemble experiments, SMFS can resolve such heterogeneities by observing one molecule at a time. Experimentally, this is realized (in solution-based SMFS) by dilution of the sample to concentrations of ˜100 pM, ensuring the diffusion of only one molecule at a time through a small observation volume (˜1 fL volume) generated by a focused laser beam. Using microscope optics and single-photon detectors, fluorescence photons are detected and analyzed.

A popular SMFS method is single-molecule FRET spectroscopy, which is the observation and measurement of FRET within a donor-acceptor pair present within a single diffusing or immobilised molecule. Such measurements allow studies of molecular interactions or structural transitions, and can resolve subpopulations or reaction intermediates.

Alternating-laser excitation (ALEX) spectroscopy is an extension of single-molecule FRET; it uses a second laser source at a wavelength that excites primarily the FRET acceptor and probes directly the presence of photoactive acceptor (i.e., an acceptor group found in a photophysical state in which it absorbs and emits light with high efficiency) in a single diffusing molecule. Alternating modulation of both lasers (typically with frequencies of 10-100 kHz, and in special cases that use interlaced pulsed lasers, up to 10-100 MHz) and detection with two spectrally separated detectors allows the skilled person to distinguish the origin of photons and to determine accurate values for FRET efficiency. Using a two-colour ALEX setup, four different photon counts, (f_{Dex}^{Dem}, f_{Dex}^{Aem}, f_{Aex}^{Dem}, f_{Aex}^{Aem}) are distinguished; the subscript describes the excitation (Dₑₓ stands for donor excitation; Aₑₓ stands for acceptor excitation) and the superscript describe the emission channel (Dₑₘ stands for donor emission; Aₑₘ stands for acceptor emission). From these values, a FRET efficiency ratio E and a stoichiometry ratio S can be calculated:

$$\begin{matrix}E = \frac{{f}_{Dex}^{Aem}}{{f}_{Dex}^{Aem} + {f}_{Dex}^{Dem}} & ( 3 ) \\ S = \frac{{f}_{Dex}^{Dem} + {f}_{Dex}^{Aem}}{{f}_{Dex}^{Aem} + {f}_{Dex}^{Dem} + {f}_{Aex}^{Aem}} & ( 4 )\end{matrix}$$

These two values are plotted in a 2-dimensional histogram, and allow the skilled person to distinguish species with different FRET (and thus proximity of the probes) as well as stoichiometry.

This is also described in International Patent application No. WO 2005/008212 which discloses the use of fluorescence spectroscopy and, in particular, FRET and ALEX spectroscopy to analyse small numbers of molecules that are present in a relatively small detection volume or zone. Information regarding physical and chemical properties of these molecules is determined by rapidly modulating the wavelength, intensity and/or polarisation of laser energy to excite fluorophores that are attached either to the molecule of interest or a molecule that interacts with the molecule of interest. Although there is mention of the use of ALEX-FRET for detection of protein-dependent coincidence of DNA, there are no details provided on how the concept will be implemented and/or how concentration can be obtained from ALEX-based histograms. Moreover, no results are reported and there is no description enabling the assay to be multiplexed and/or probe transcription factor concentrations in cells to be determined.

We have now surprisingly found a method using single-molecule fluorescence spectroscopy for the detection of DNA-binding proteins and related ligands. In short, we demonstrated the following: protein-dependent DNA coincidence to detect DNA-binding proteins in dilute solutions; multiplexing of our assay by detecting recognize two DNA-binding proteins simultaneously in the same solution; compatibility with complex biological samples; and sensing of changes in gene expression in cells. These results are summarised in the figures accompanying this disclosure. We also describe the concepts that will substantially increase the multiplexing capability of the assay and allow real-time monitoring of DNA-binding proteins in living cells.

Thus, according to a first aspect of the invention we provide a method of detection of the protein-dependent coincidence of DNA in a sample which comprises detection using luminescence of one or more luminophores introduced into DNA with one, two or more DNA fragments which fragments are bound using one or more DNA-binding proteins.

This capability is used to determine the concentration of proteins and other analytes related to binding capabilities of proteins.

The luminescence detection as hereinbefore described is preferentially, single molecule fluorescence spectroscopy. The fluorescence technique may or may not comprise the use of alternating-laser excitation ALEX and/or FRET. A particular embodiment comprises the use of ALEX-FRET. Other embodiments do not require either use of ALEX or use of FRET.

As hereinbefore described, it is an advantage of the present invention that the method is general enough to be used in either solution-based format (i.e. it does not rely upon the use of a solid state array) or a surface-based format (by anchoring one of the detection reagents on a solid support). Therefore, we especially provide solution-based methods of detection of [or determining] the protein-dependent coincidence of DNA as hereinbefore described. We also describe an example of using the assay on a solid support.

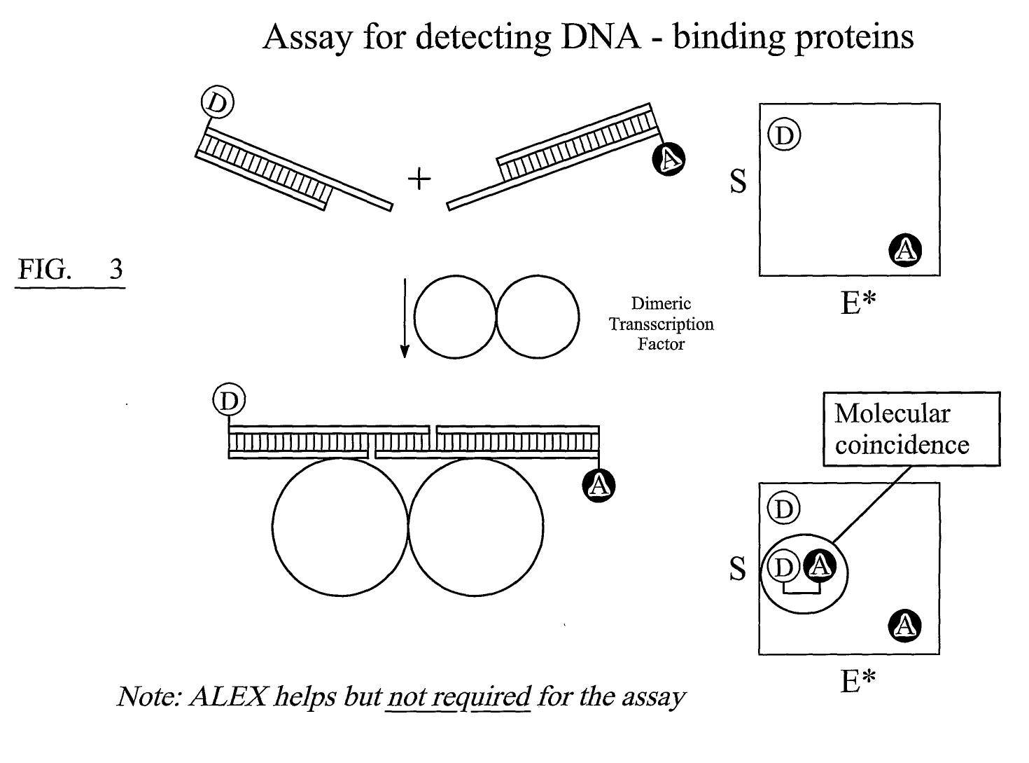

For the purposes of our assay, the fluorophores can be placed far from the protein binding site (as long as the fluorophores are spaced by less than approximately 200 base pairs); as such, there is no perturbation of protein-DNA binding and no information about the exact structure of the protein-DNA complex is necessary. The concept is described in FIG. 3. In the absence of a DNA-binding protein, for example, a dimeric protein, such as a dimeric transcription factor, two DNA fragments with short and complementary single-stranded DNA tails diffuse independently in solution (top panel). In the presence of a DNA-binding protein that binds specifically to the fully assembled DNA site, the two DNA fragments diffuse as a single molecular complex (a 1:1 donor-acceptor species); such a species can be easily differentiated from the free DNA half-sites and can be counted, giving information about the concentration of the specific DNA-binding protein.

Therefore according to a further aspect of the invention we provide a method as hereinbefore described wherein the determination includes obtaining the concentration of one or more DNA-binding proteins.

The advantages of the method include, inter alia:

The assay allows measurements of low concentration of DNA-binding proteins. The assay can easily detect concentrations of less than 1 protein copy per bacterial cell (˜1 nM) and is compatible with measurements down to 10-20 pM (concentration of 1 protein copy in a eukaryotic nucleus). Overall, the single-molecule method allows probing of the interactions using 1000-fold lower concentrations and 100-fold smaller volumes compared to the molecular-beacon approach in solution; this reduces the cost for reagents and minimises the sample volume needed for diagnostic assays or drug-discovery assays, paving the way for high-throughput screening assay formats.

We note that the capability of detecting DNA-binding proteins does depend to a certain degree to the affinity of the proteins for their binding site. For example, detecting proteins with K_{d} values >5 nM requires operating at concentrations higher than the ones used for typical single-molecule measurements (i.e., ˜100 pM). This can be done using fluorescence cross-correlation spectroscopy and confinement of molecules in nanostructures (e.g., nanochannels, nanopores, nanowells, and nanopipets). Alternatively, optimisation of solution conditions for protein-DNA binding (e.g., by decreasing salt concentration and by using macromolecular crowding agents) can reduce the K_{d} and allow sufficient number of positive events to be recorded, leading to protein detection and quantitation.

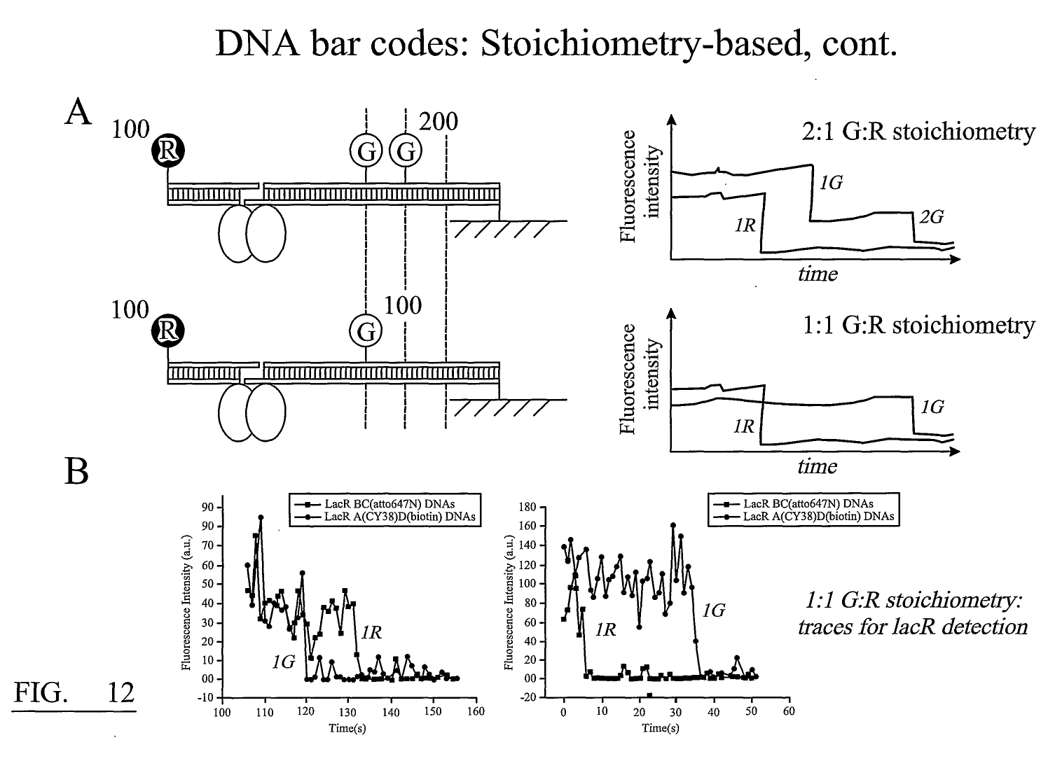

The assay is compatible with measurements on immobilised molecules. In that embodiment, one DNA half-site is attached to a solid support (e.g., a modified glass or quartz surface) and the other half-site is added in solution. In the presence of the transcription factor specific for the DNA pair, the fluorophores on the half-site co-localise; this co-localisation can be detected using dual- or alternating-laser excitation and imaging of the surface on an sensitive CCD camera. This embodiment has been exemplified with the detection of a bacterial transcription factor. The long collection-time per molecule increases the signal-to-noise ratio of the measurement and its sensitivity; moreover, since 100-1000 molecules can be sampled in a few seconds, this format affords a very high-throughput. Finally, the ability to step-wise photobleach fluorophores (FIG. 12) provides an elegant and direct way to count the number and identity of fluorophores placed on either of the two DNA half-sites, creating opportunities for multiplexing; reports in the literature have clearly demonstrated counting up to 6 identical fluorophores on the same immobilised single molecule by step-wise photobleaching, an irreversible photodestruction process that causes step-wise decreases in the amount of fluorescence emitted by a single immobilised fluorescent molecule.

Concentration determination is possible by a simple measurement of the fraction of assembled DNA half-sites which is measured directly from the ratio of [D-A]I[A-only] species. A specific site will have a specific dynamic range for concentration determination, which will depend on the Kd for the protein-DNA interaction. However, it is trivial to increase the Kd of the interaction (e.g., by one or more basepair substitutions in the DNA site or by increasing the ionic strength of the binding and observation buffers) to address detection at higher concentration of the specific transcription factor. This calculations can be adjusted to account for trivial effects that convert a D-A species into A-only species such as donor-photobleaching, incomplete labelling of DNA with donor and acceptor fluorophores, and dissociation of the complex during the data acquisition at pM concentrations.

Although a FRET observable can help multiplexing by adding a tunable observable, the assay is FRET-independent; this fact increases the generality of the method since it removes the requirement for precise knowledge of the molecular details and the crystal structure of the protein-DNA complex in question.

The method of the present invention allows extensive multiplexing for detecting several proteins without separation steps and without the use of arrays or solid supports. This is based on the use of DNA “bar codes”. In short, use of multi-colour SMFS to detect 3 (e.g., Blue, Green and Red) or 4 (e.g., Blue, Green, Red and Infra-Red) spectrally distinct fluorophores on a single diffusing or immobilized molecule permits multiplexing based on multi-dimensional histograms. The histograms can be constructed using ratiometric expressions such as the E-S histogram in ALEX-FRET. For example, use of 3 spectrally distinct fluorophores separated by different distances for each DNA corresponding to a different transcription factor can achieve extensive multiplexing by leveraging the FRET dimension of the E-S histograms. This is achieved by using DNA fragments having distinct and resolvable sets of interprobe distances that result in distinct 3-dimensional FRET histograms.

Moreover, use of 3 or 4 spectrally distinct fluorophores and brightness levels can achieve extensive multiplexing by at least two ways. In a first embodiment, the use of 3 or 4 spectrally distinct fluorophores leverages both dimensions of the E-S histograms.

In a second embodiment, the use of 3 or 4 spectrally distinct fluorophores can result in new multi-dimensional histograms based on new ratiometric expressions; importantly, this does not require either use of ALEX or use of FRET. For example, simultaneous triple-laser excitation of 3 spectrally distinct fluorophores (e.g., Blue, Green and Red, hereafter B, G, and R respectively) allows definition of the following fluorescence-intensity ratios:

GB ratio=Gₑₘ/Bₑₘ
RB ratio=Rₑₘ/Bₑₘ

The best embodiment of the triple-labeling scheme uses a B probe on one half-site (that allows observation of assembly of a full DNA site) and a G-R combination on the other half-site (that allows FRET- or stoichiometry-based coding for the specific protein that assembles the full DNA site). To increase multiplexing, the assay requires DNAs with combinations of different levels of Gₑₘ and Rₑₘ. For example, if 5 distinct intensity levels are generated for Gₑₘ and 5 distinct intensity levels are generated for Rₑₘ, a total of 5²=25 combinations are possible, coding for 25 different DNA-binding proteins. Fluorescent DNAs with different levels of Gₑₘ and Rₑₘ can be achieved either by introducing different numbers of copies of spectrally distinct fluorophores or by using spectrally identical fluorophores with substantially different brightness.

Multiplexing can also be leveraged by combining FRET and stoichiometry ratios with the observable of fluorescence lifetime, which can be obtained through the use of pulsed lasers and the measurement of precise timing of the photon-arrival time of an emitted photon in relation to a laser-excitation pulse.

Multiplexing information about the presence and the mutational or modified status of a transcription factor. DNA-binding proteins are often mutated in diseased states or are modified (e.g., phosphorylated) as a part of their biological function. A fluorescently labelled antibody that can sense mutation or modification of a DNA-binding protein can report on its mutational or otherwise modified status. This way, one can detect that a transcription factor is present in a biological fluid [by observing DNA binding to a diffusing protein molecule] and that it is phosphorylated [by observing antibody binding to the phosphorylation epitope on the SAME diffusing protein molecule].

Overall, in different embodiments, the multiplexing assay is compatible with single-laser excitation, dual-laser excitation, triple-laser excitation, quadruple-laser excitation, ALEX, and FRET; the different embodiments provide different levels of accessible information, extent of multiplexing and useful concentration range.

We envisage that generation of several, clearly resolvable DNA bar codes will be useful in other contexts, e.g., coding for short DNA sequences that help identify complementary sequences during single-molecule DNA sequencing reactions, as well as coding for antibodies directed to specific proteins.

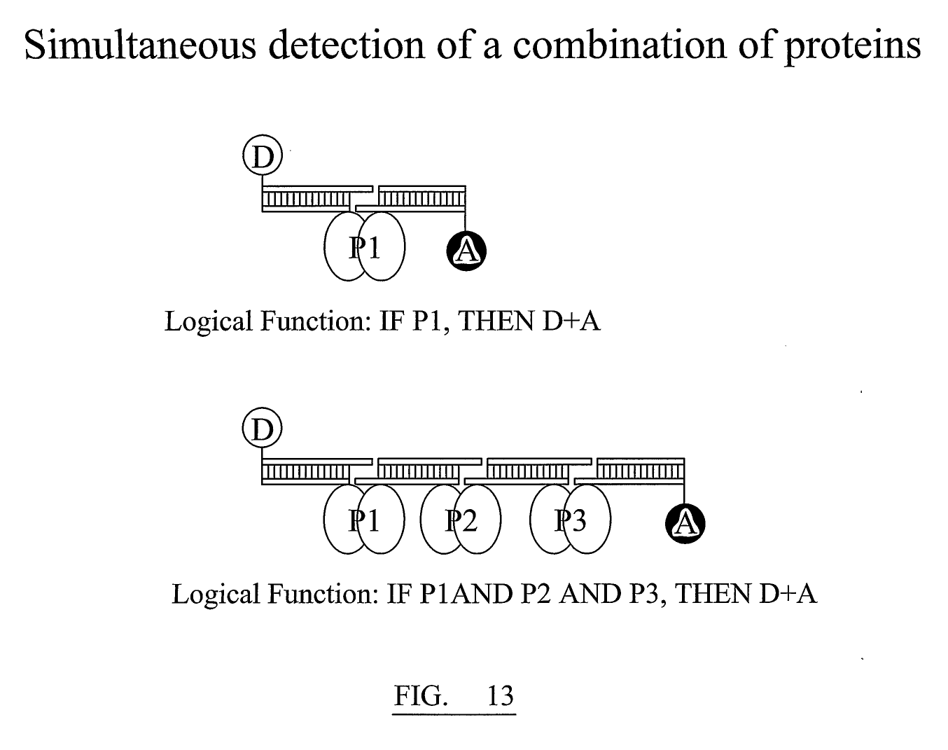

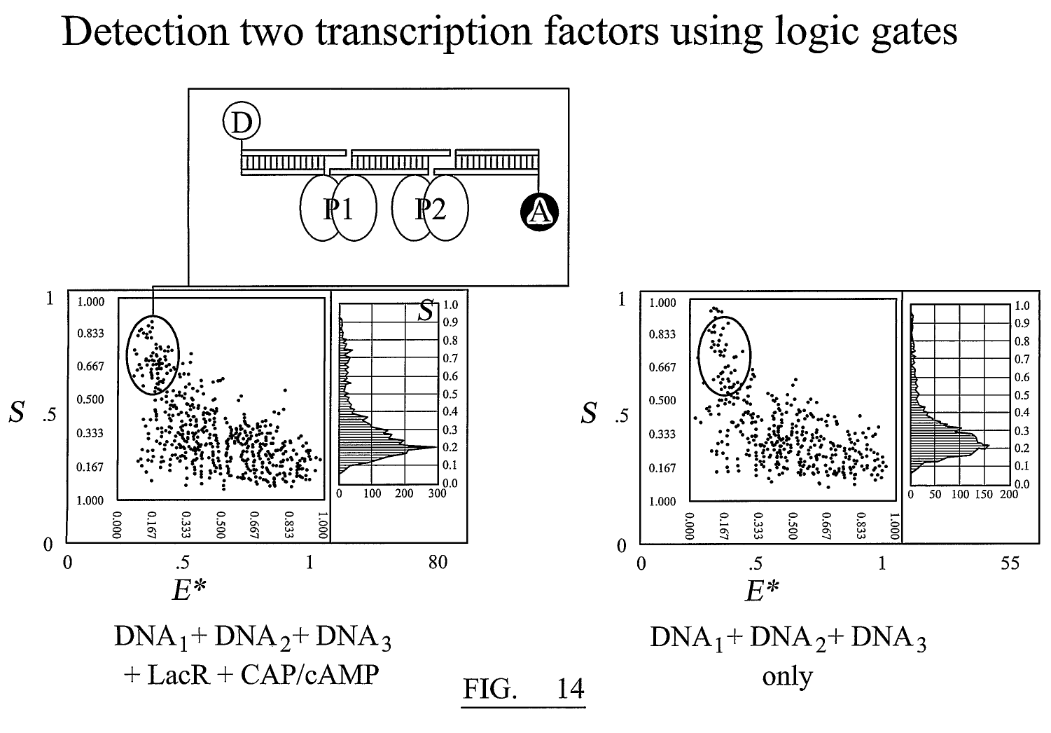

We envisage building logical functions based on the presence of DNA-binding proteins and ligands that modulate their activity. For example, the 3 individual DNA-binding sites for 3 separate DNA-binding proteins can be designed in a way that results in an observable of molecular coincidence only when all 3 proteins in question are present in solution (FIG. 13). The concept has been exemplified for two bacterial transcription factors (FIG. 14). This format minimizes the complexity and cost of reagents needed to detect a certain profile of DNA-binding proteins and related ligands.

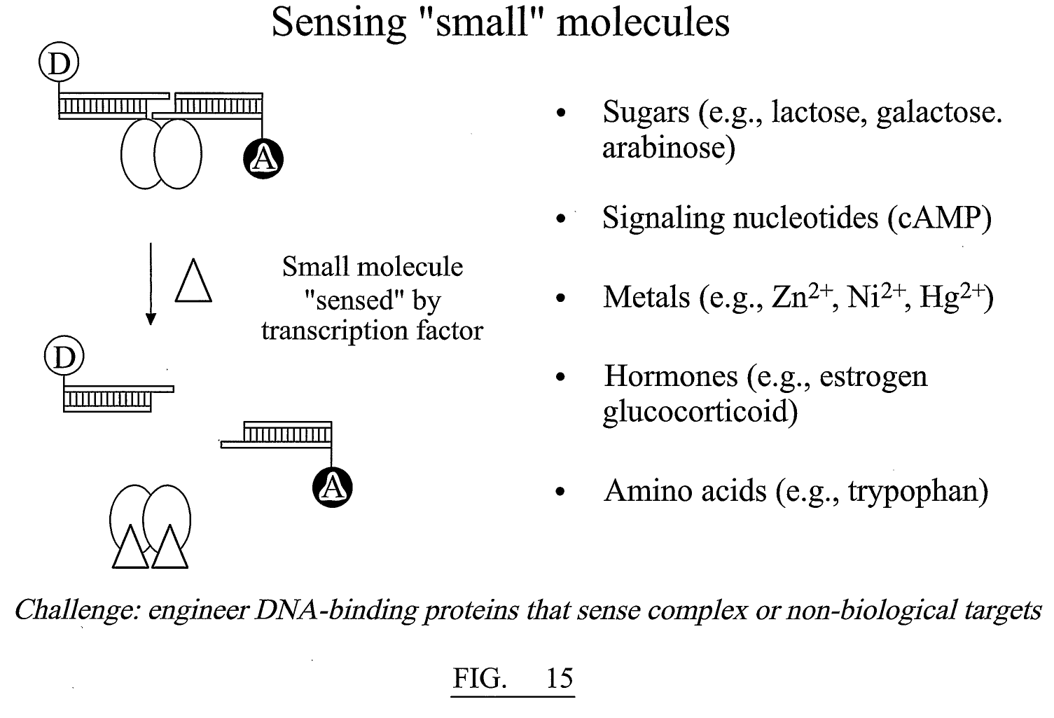

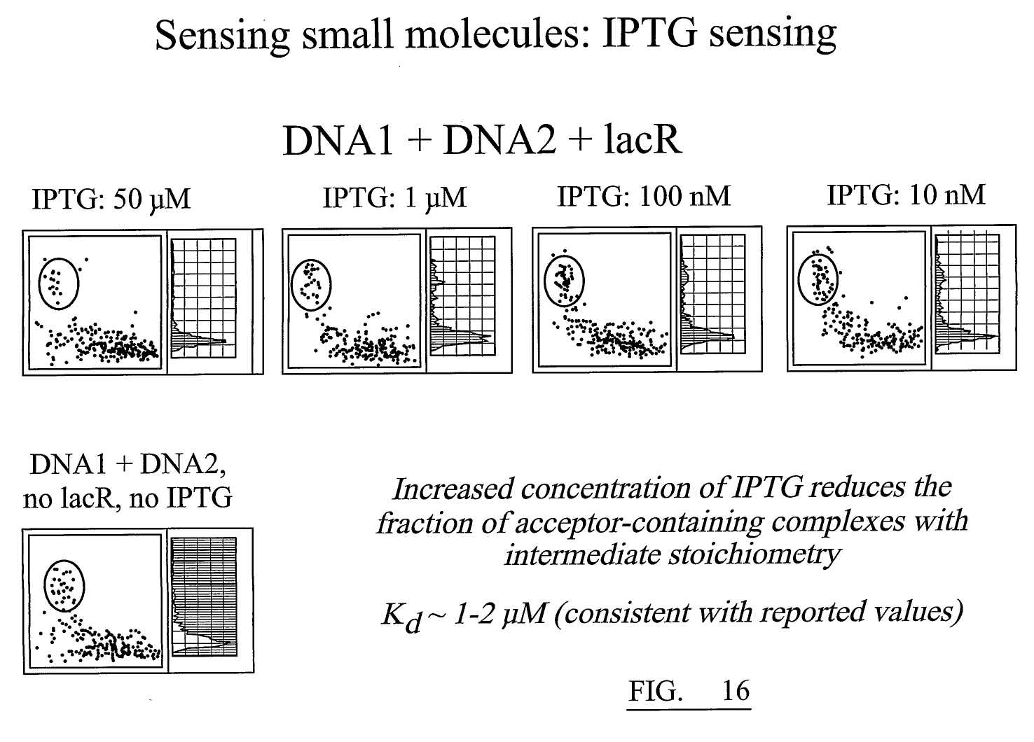

Since transcription factors act as sensors for smaller molecules and important metabolites such as sugars, nucleotides, metals, hormones, and amino acids (FIG. 15), we envisage that the multiplexing assay will be able to detect simultaneously the presence and concentration of many small molecules sensed by DNA-binding proteins. The concept has been exemplified for the detection of a small molecule that is recognised by a bacterial transcription factor (FIG. 16).

We further provide an assay which comprises the detection of the protein-dependent coincidence of DNA in a sample which comprises detection using luminescence detection of DNA with at least two DNA fragments which fragments are bound using a DNA-binding protein and the determination of analyte concentration.

Quantum dots (QDs) are special colloidal semiconductor luminescent nanoparticles converted into novel biological labels; the unique properties of QDs have created new opportunities for sensitive and extended observations of biological processes in living cells. QDs are 5-10 times brighter than organic fluorophores and are extremely photostable: while organic fluorophores usually photobleach in seconds upon exposed to excitation intensities typical for single-molecule fluorescence experiments, QDs are stable for tens of minutes. The photostability of QDs extends the observation of processes in cells and enables in vivo detection of DNA-binding proteins using the DNA biosensor concept.

The unique spectral properties of QDs should allow implementation of the protein-dependent coincidence assay without the need for ALEX, without any modulation, and without FRET. This is due to the fact that each DNA half site can contain a QD excitable by a single wavelength (e.g., 488 nm) whereas each QD emits at a distinct wavelength.

The capability of detecting low-abundance protein within cells will also be important in the case that the population of cells is heterogeneous, with a small fraction of the cells expressing a protein that may signify a diseased state.

The invention will now be illustrated by way of example only and with reference to the accompanying figures:

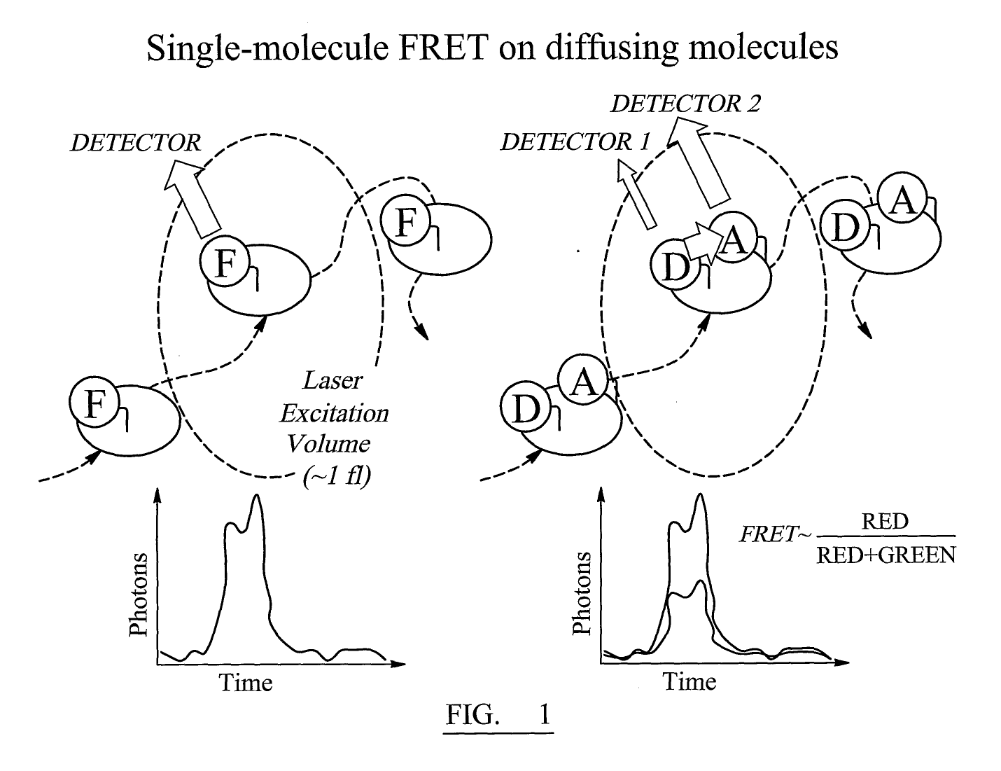

FIG. 1 (prior art) is a schematic representation of single-molecule FRET on diffusing molecules. In order to perform FRET at the single molecule level, we use confocal microscopy. We allow a fluorescent molecule to flow through a tiny excitation volume (defined by the confocal optics of a fluorescence microscope). When the molecule enters the excitation volume, it is excited by the laser, and emits a photon. This excitation/emission cycle is repeated thousands of times, and the resulting photons are detected. When the number of photons is plotted as a function of time, the single molecule appears as a “burst” of fluorescence on dark background. If there is an acceptor in close proximity, some energy is transferred to the acceptor, and it is detected as light of longer wavelength. Information about the D-A distance can be obtained by a ratio of these signals.

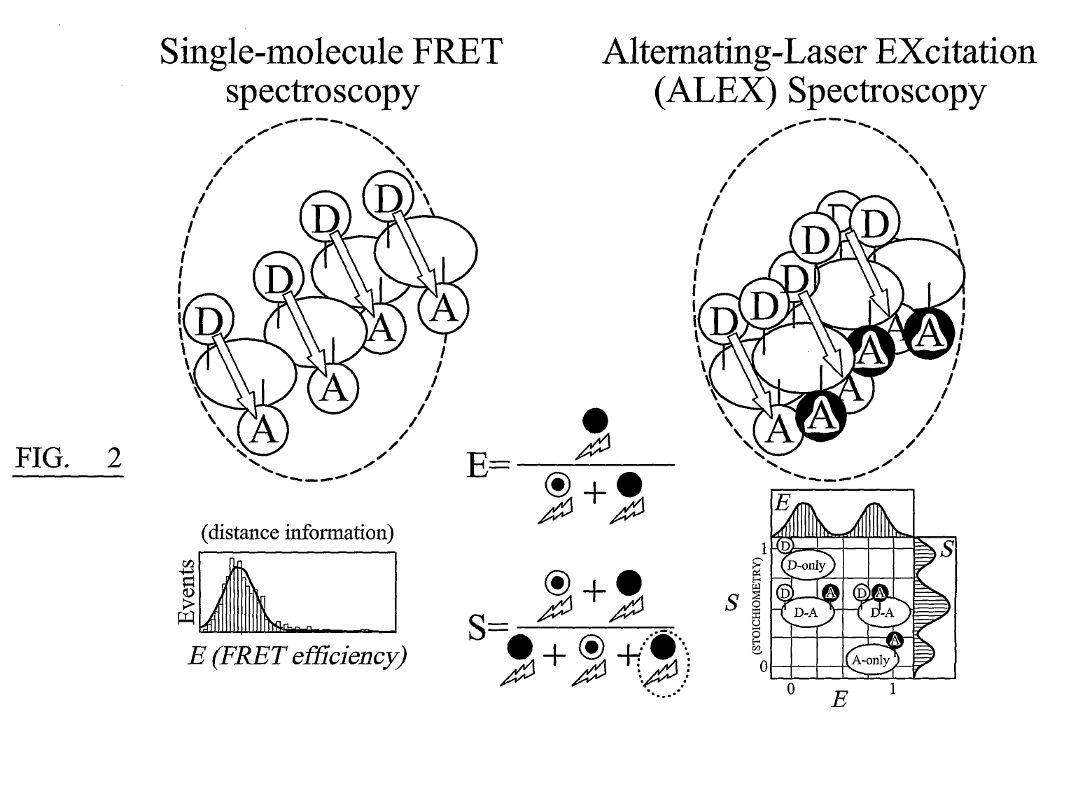

FIG. 2 (prior art) is a schematic representation of single-laser excitation FRET versus ALEX.

FIG. 3 is a schematic representation of the use of ALEX and DNA fragments for detecting DNA-binding proteins. In the absence of a dimeric transcription factor, two DNA fragments with short and complementary single-stranded DNA tails diffuse independently in solution (3a). In the presence of a dimeric transcription factor that binds specifically to the fully assembled DNA site, the two DNA fragments diffuse as a single molecular complex (a Donor-Acceptor species); such a species can be easily differentiated from the free DNA half-sites and can be counted, giving information about the concentration of the specific DNA binding protein.

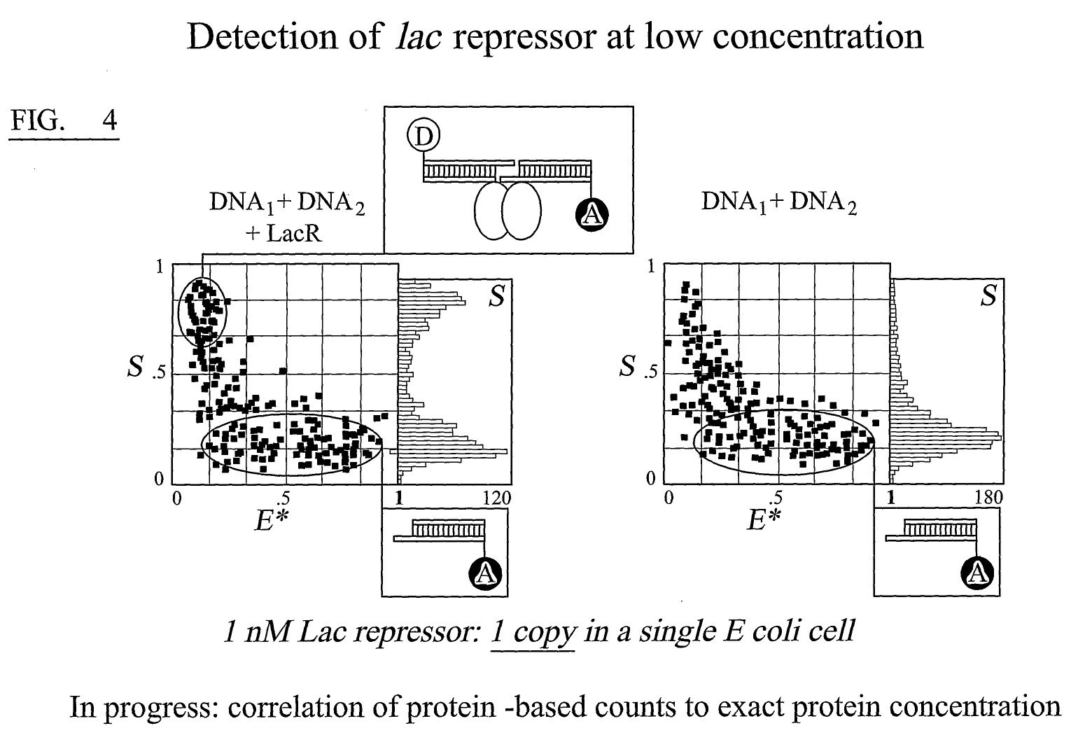

FIG. 4 is a schematic representation demonstrating the detection of a transcription factor that represses transcription. Left panel: in the presence of lac repressor (a tetrameric DNA-binding protein that represses gene transcription in bacteria), protein-specific DNA fragments give rise to a population with S˜0.8 and E*˜0.15; this population corresponds to the complex of DNA fragments with the lac repressor protein (as shown in the orange rectangle). Right panel: in the absence of lac repressor, only a few counts due to random coincidence are present in the area previously occupied by the protein-DNA complex.

The results that demonstrate that the validity of the concept are in FIG. 4 (see Fig. legend). In the presence of the transcription factor lac repressor, an additional population appears in the E-S histogram generated by ALEX. Positive signals have been obtained down to 300 pM lac repressor concentration, which shows that our method is sensitive to less than 1 copy of protein in a bacterial cell.

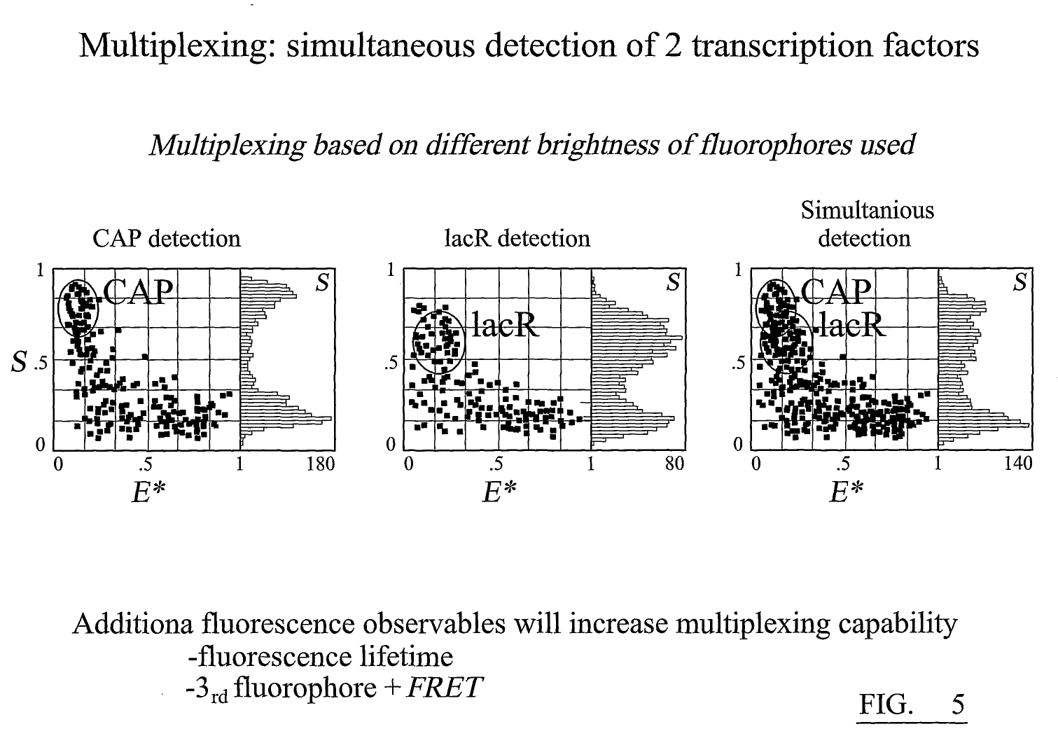

FIG. 5 is a schematic representation demonstrating multiplexing using DNA fragments labelled with fluorophores of different brightness. Left panel: in the presence of CAP (a dimeric DNA-binding protein that activates gene transcription in bacteria), protein-specific DNA fragments give rise to a population with S-0.8 and E*0.15; this population corresponds to the complex of DNA fragments with CAP. Middle panel: in the presence of lacR, different protein-specific DNA fragments (with the same acceptor fluorophore but with a donor fluorophore less bright than the donor fluorophore used in the case of CAP) give rise to a population with S˜0.6 and E*˜0.2; this population corresponds to the complex of DNA fragments with lacR. Right panel: in the presence of both proteins, two peaks are obtained corresponding to the two individual complexes and to the presence of both DNA binding proteins in the examined solution.

By preparing two pairs of DNA fragments (one pair for transcription factor lac repressor and one pair for transcription factor catabolite activator protein), we have demonstrated that we can detect two transcription factors in the same solution, without the use of multi-well plates or separation steps. The observable responsible for resolving CAP-DNA complexes from lacR-DNA complexes depends on the difference in the molecular brightness of FRET donor D used for the left half site of CAP vs the left half-site of lacR; this difference in molecular brightness (summarized as photon count f_{Dex}^{Dem} in equation 4) affect the stoichiometry parameters S, resulting in different S values for the two protein-DNA complexes.

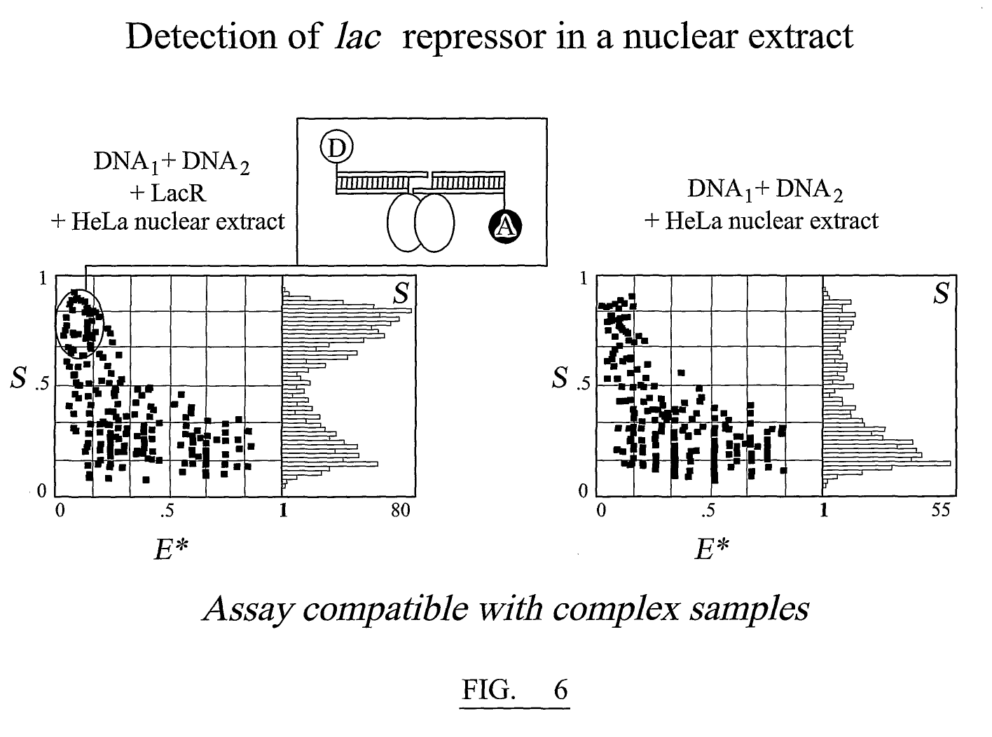

FIG. 6 is a schematic representation demonstrating detection of a transcription factor in a complex biological fluid. Left panel: in the presence of lac repressor in a nuclear extract prepared from eukaryotic cells (HeLa cells), protein-specific DNA fragments give rise to a population with S˜0.7 and E*˜0.15; this population corresponds to the complex of DNA fragments with the lac repressor protein. Right panel: in the absence of lac repressor, only a few counts due to random coincidence and possible to protein(s) similar to lacR are present in the area previously occupied by the protein-DNA complex.

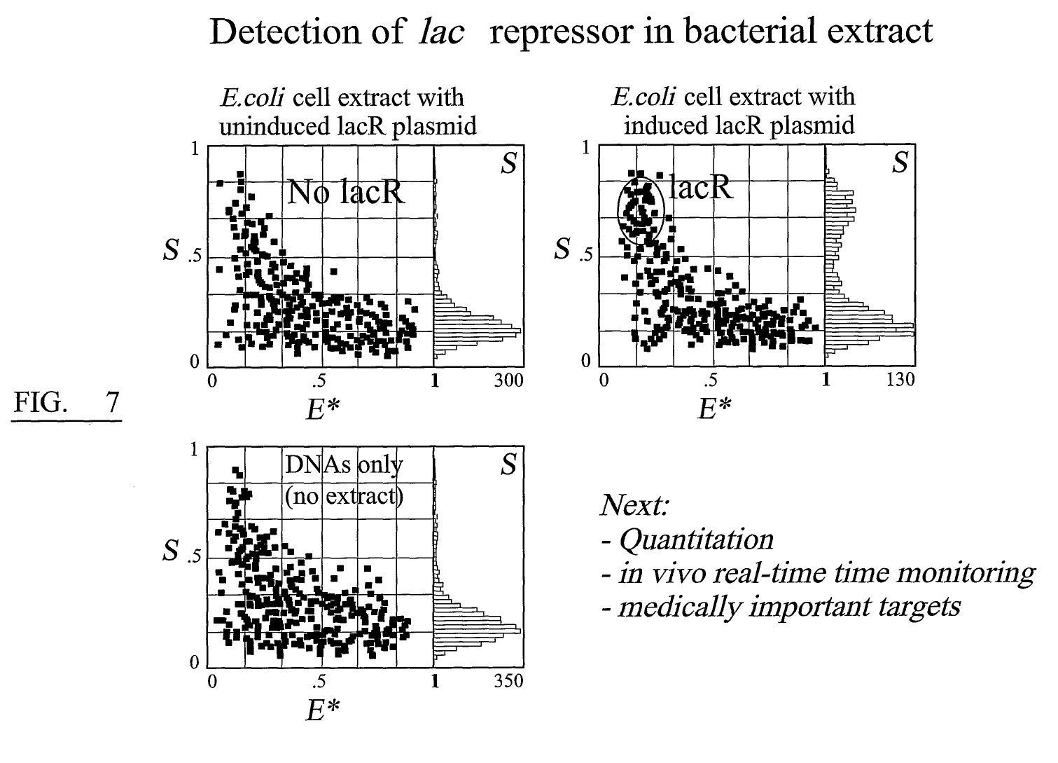

FIG. 7 is a schematic representation demonstrating detection of a transcription factor in a bacterial extracts after switching on its gene expression. Left panels: in cellular extracts from bacterial cells in which the gene for a transcription factor is switched off, no lacR is observed as compared to a control experiment where only DNA fragments (and no cellular extract) have been used. Right panel: if the gene for the production of lac repressor is switched on, then protein-specific DNA fragments give rise to a population with S˜0.7 and E*˜0.15; this population corresponds to the complex of DNA fragments with the lac repressor protein.

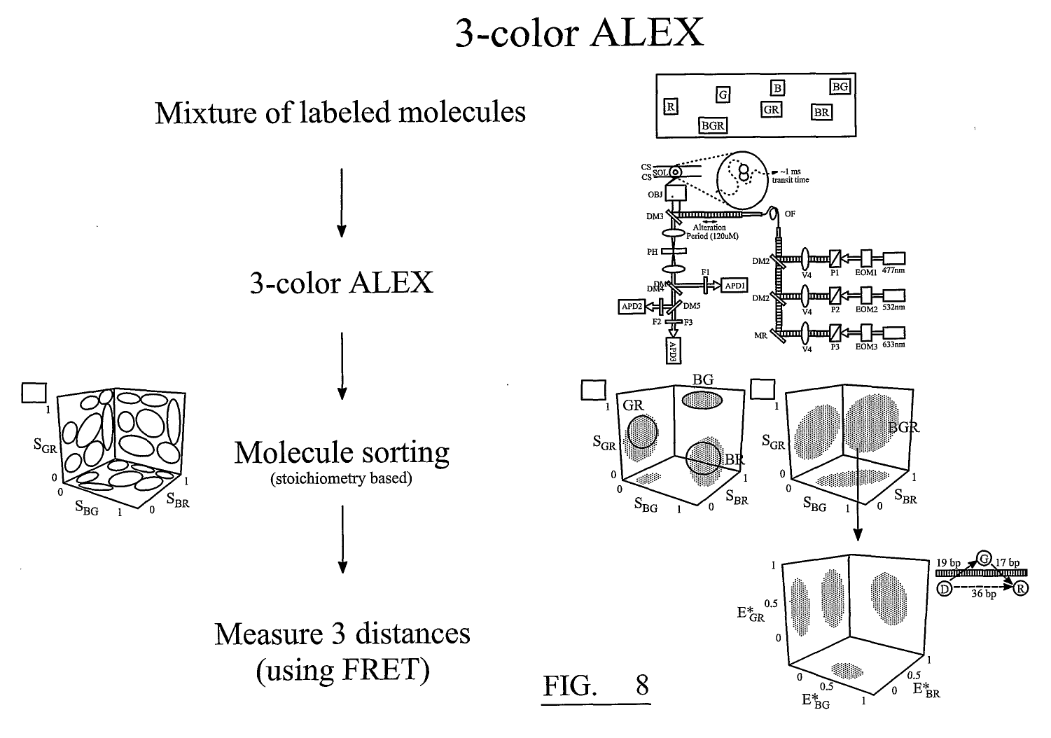

FIG. 8 (prior art) is a schematic representation illustrating the principle of 3-colour ALEX.

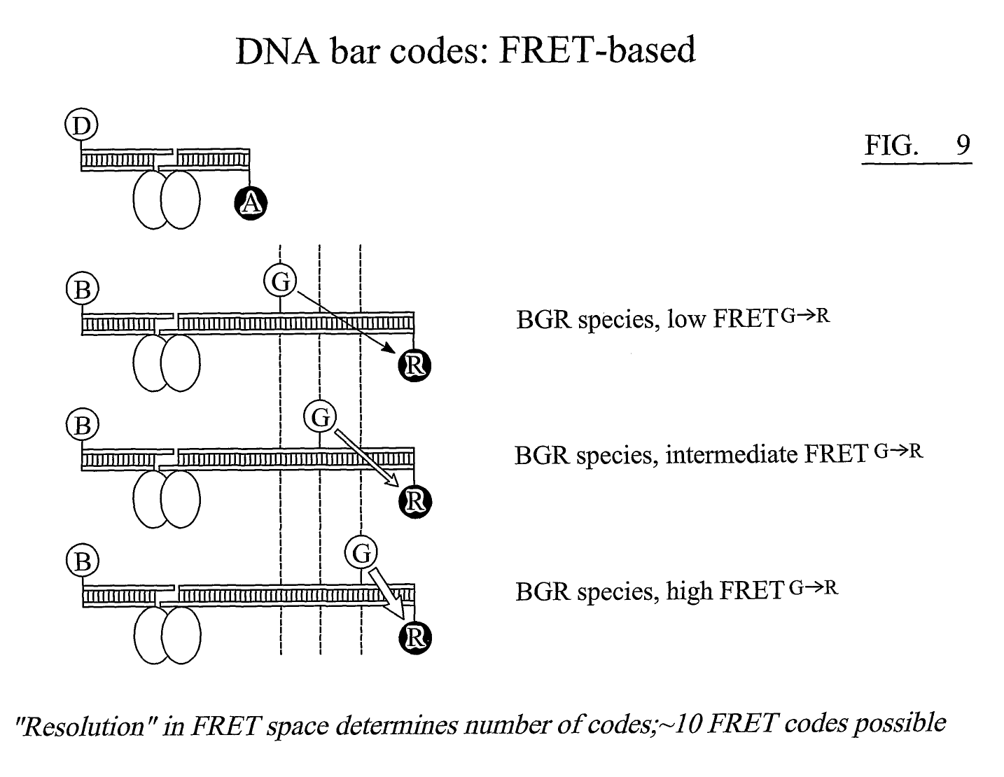

FIG. 9 is a schematic representation demonstrating the principle of FRET based DNA bar coding for multiplexing (BGR representing Blue, Green and red respectively).

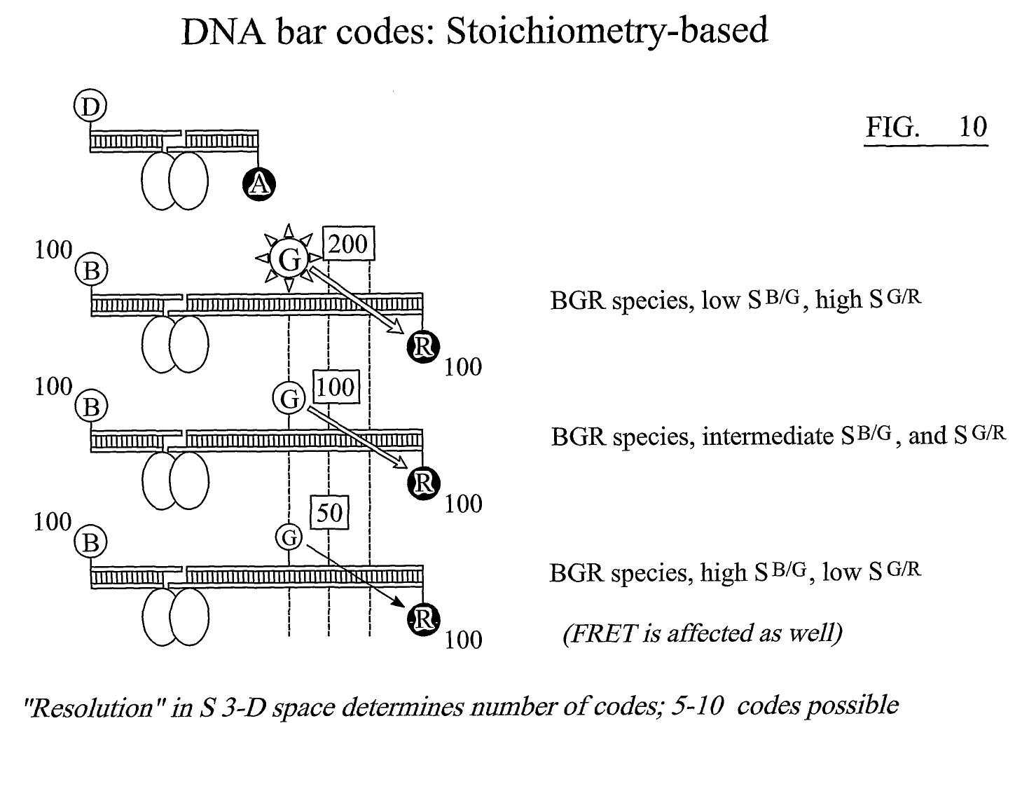

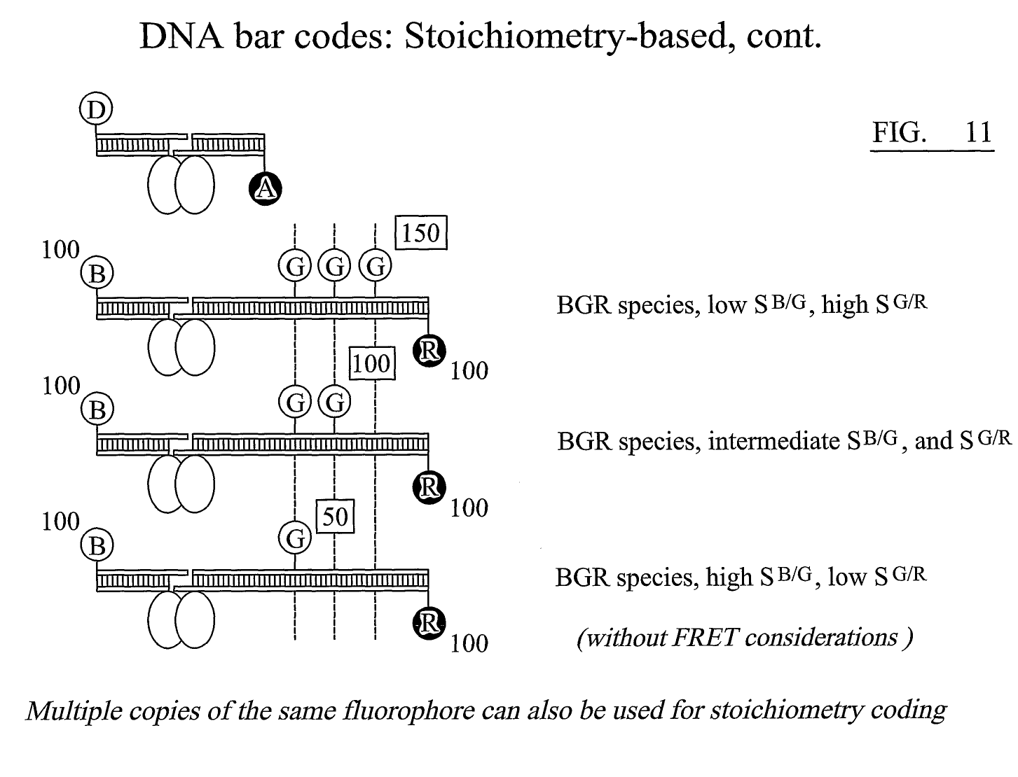

FIGS. 10 and 11 are schematic representations demonstrating the principle of stoichiometry based DNA bar coding for multiplexing.

FIG. 12 panel A is a schematic representation demonstrating multiplexing performed by measuring the stoichiometry of immobilised molecules by stepwise photobleaching. FIG. 12 panel B shows two representative fluorescence intensity time-traces with two photobleaching steps (one for a green fluorophore and one for a red fluorophore) for a full lacR-assembled DNA site immobilised on a solid support. The photobleaching pattern points to a 1:1 G:R fluorophore stoichiometry.

FIG. 13 is a schematic representation of the simultaneous detection of the presence of multiple DNA binding proteins in a solution.

FIG. 14 is a schematic representation of the simultaneous detection of the presence of two DNA binding proteins in a solution. Molecular coincidence is observed only in the presence of both CAP(+cAMP) and lacR (left panel).

FIG. 15 is a schematic representation demonstrating sensing small molecules. An example of sensing the concentration of a small molecule using protein-dependent DNA coincidence. In the absence (or low concentration) of the molecule to be sensed (yellow triangle), the transcription factor binds to DNA with high affinity and gives rise to DNA coincidence detected by single-molecule fluorescence. In the presence (or high concentration) of the molecule to be sensed, the transcription factor is converted to a conformation that does not bind to DNA and this leads to disappearance of the previously detected population that arose due to DNA coincidence.

FIG. 16. Sensing a lactose analog (IPTG) using DNA biosensors. In the presence of low concentration of IPTG, lacR does bind and assemble the full site, increasing the relative fraction of bursts with intermediate stoichiometry; in the presence of high concentration of IPTG, lacR does not assemble the full site, decreasing the relative fraction of bursts with intermediate stoichiometry. A plot of the number of molecules with intermediate stoichiometry can be used as a calibration curve to identify the concentration of the small molecule, provided that the concentration is not much higher or much lower than the Kd for the interaction of the small molecule with the DNA-binding protein.

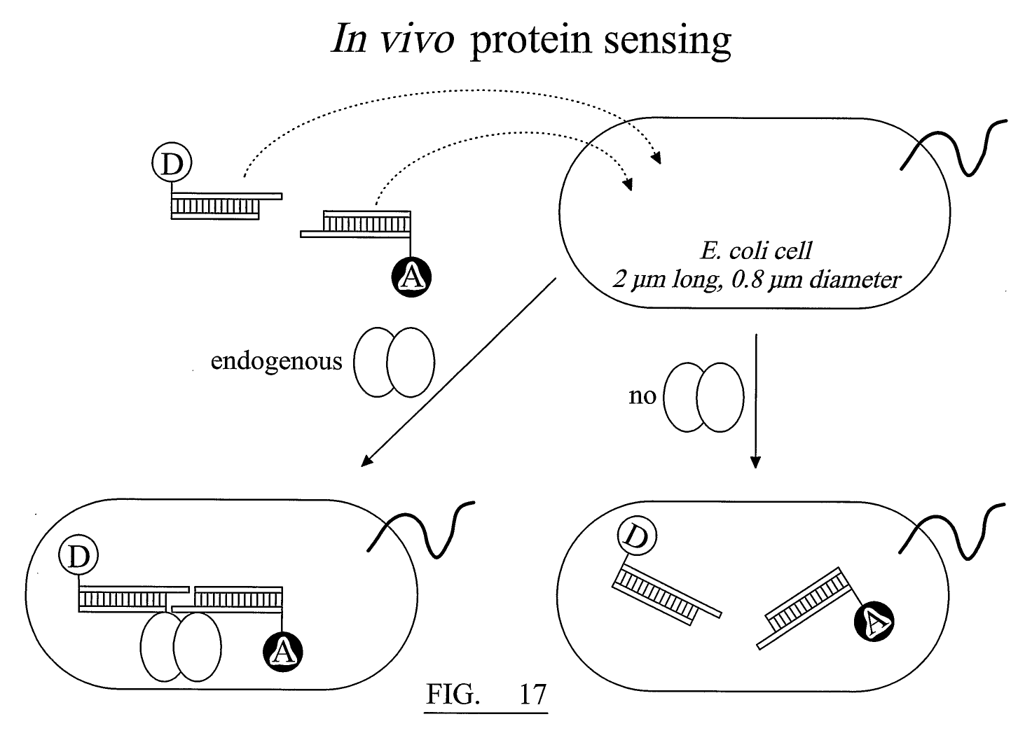

FIG. 17 is a schematic representation demonstrating detection of transcription factors in vivo using DNA biosensors.

1. A method of detection of the protein-dependent coincidence of DNA in a sample which comprises detection using luminescence of one or more luminophores introduced into DNA with one, two or more DNA fragments which fragments are bound using one or more DNA-binding proteins.

2. A method according to claim 1 wherein the one or more luminophores are fluorophores.

3. A method according to claim 2 wherein the luminescence detection is single molecule fluorescence spectroscopy.

4. A method according to claim 1 wherein the luminescence detection is proximity-based fluorescence.

5-8. (canceled)

9. A method according to claim 4 wherein the fluorescence technique comprises cross-correlation spectrosopy.

10. A method according to claim 9 wherein the DNA binding protein is confined in a nanostructure.

11-17. (canceled)

18. A method according to claim 1 wherein the fluorophores are spaced apart by up to 200 base pairs.

19. (canceled)

20. A method according to claim 1 wherein the sample comprises at least two different fluorophores.

21. A method according to claim 1 wherein the method includes detecting/measuring the concentration of one or more of the DNA-binding proteins.

22. A method according to claim 1 wherein the one or more of the DNA-binding proteins is a multimeric transcription factor.

23-27. (canceled)

28. A method according to claim 1 wherein the method comprises multiplexing for detecting several proteins.

29. A method according to claim 1 wherein the method does not include separation steps.

30. (canceled)

31. A method according to claim 1 wherein the method is capable of detecting 3 or more fluorophores on a single diffusing or immobilized single molecule.

32-39. (canceled)

40. A method according to claim 1 wherein the method includes simultaneous detection of a combination of proteins.

41-45. (canceled)

46. A method according to claim 1 wherein transcription factors act as sensors for transcription factor p53.

47. (canceled)

48. A method according to claim 1 wherein the one or more luminophores are colloidal semiconductor nanoparticles (quantum dots).

49. (canceled)

50. A method of detection of the protein-dependent coincidence of DNA in a sample which comprises detection using fluorescence of one or more colloidal semiconductor nanoparticles introduced into DNA.

51. An assay for the detection of the protein-dependent coincidence of DNA in a sample which comprises detection using luminescence detection of DNA with at least two DNA fragments which fragments are bound using a DNA-binding protein.

52. A sensor for the detection of the protein-dependent coincidence of DNA in a sample which sensor utilises a method according to claim 1.

53. (canceled)

54. A sensor for the detection of the protein-dependent coincidence of DNA in a sample which sensor utilises a method according to claim 50.

55-58. (canceled)
